(19) United States
(12) Patent Application Publication
(10) Pub. No.: US 2026/0224367 A1
(43) Pub. Date: Aug. 6, 2026

(54) HEXALOBULAR IMPLANT

(71) Applicant: Beacon Biomedical, LLC, Jupiter, FL (US)

(72) Inventors: Abdul Abu Baker, McKinney, TX (US); Daniel D. McPhillips, Ham Lake, MN (US); Jonathan M. Lewis, Palm Beach Gardens, FL (US)

(21) Appl. No.: 19/386,382

(22) Filed: Nov. 12, 2025

Related U.S. Application Data
(60) Provisional application No. 63/719,582, filed on Nov. 12, 2024.

(51) Int. Cl. A61F 2/30; A61F 2/46

(52) U.S. Cl. CPC A61F 2/30771; A61F 2002/30171; A61F 2002/30784; A61F 2002/30828; A61F 2002/30841; A61F 2002/3092; A61F 2002/30995; A61F 2002/4629

(57) ABSTRACT

Spinal implants for use in fusion procedures, such as sacroiliac fusion procedures, are provided. The implant provides mechanical stability when inserted into an anatomical structure(s) until fusion occurs. In certain embodiments, the implant may comprise a multi-surfaced, cross-sectional configuration, such as a 6-starred shaped (hexalobular) cross-sectional configuration, and radially dispersed longitudinal splines. When used for sacroiliac fusion procedures, the implant is configured to prevent sacroiliac joint spreading.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 63/719,582, entitled “HEXALOBULAR IMPLANT”, filed Nov. 12, 2024. The contents of the above referenced application are incorporated herein by reference in their entirety.

### FIELD OF THE INVENTION

The present invention relates to surgical devices and procedures; to surgical implants relating to fusion procedures; and more specifically, to a bone implant used in sacroiliac fusion procedures.

### BACKGROUND OF THE INVENTION

The sacroiliac joint is situated between the sacrum and the ilium of the pelvis. Humans have two sacroiliac joints in their lower back on each side of the spine, each of which can be a source of pain and disease state. The sacroiliac joint is a critical anatomical structure and plays a pivotal role in providing stability and support to the human spine. It is essential for transmitting forces between the upper body and the lower extremities. The sacroiliac joint acts as a connecting link, helping to distribute loads and facilitating movement while ensuring the sacrum and ilium maintain their relative positions. The sacroiliac joint forms a crucial component of the body's biomechanical system and its proper functioning is essential for maintaining posture, balance, and mobility.

Various ailments can be attributed to problems with the sacroiliac joint. Pain that originates from the sacroiliac joint can manifest in various forms, including localized discomfort, radiating pain into the lower back, buttocks, or down the legs, and in some cases, it may even mimic symptoms of lumbar spine issues. Sacroiliac pain can be a result of arthritis, traumatic injury, pregnancy and post-partum, systemic inflammatory conditions, and infection. Prior to surgical intervention, treatment for sacroiliac joint pain often involves physical therapy, pain medications, injections or bracing with a belt. If such treatments are not effective, surgical intervention may be required. One surgical approach in alleviating sacroiliac joint pain is sacroiliac joint fusion. Sacroiliac joint fusion results in immobilization of the joint, thereby reducing or eliminating pain by eliminating motion at the sacroiliac joint itself.

Sacroiliac joint fusion is a surgical intervention designed to stabilize the sacroiliac joint and is performed via an anterior approach, a posterior approach, or a lateral approach. Sacroiliac joint fusion offers several advantages over conventional open surgical procedures. Unlike open surgeries, which often require general anesthesia, extensive operative time, extended hospitalization, and significant post-operative pain, sacroiliac joint fusion is a minimally invasive procedure. It can often be performed under local anesthesia or sedation, requires shorter operative times, minimizes recovery periods, and can be done on an outpatient basis. This approach significantly reduces the physical and financial burdens on patients while delivering effective pain relief and joint stability.

While sacroiliac joint fusion represents a major advancement in treating sacroiliac joint pain and associated diseases or conditions, there remains a need for further improvements, particularly in the realm of implant devices used during the procedure. Traditional implants often employ bone screws, anchors, rods, bands, and plates to stabilize the joint. However, these implants are not without their shortcomings. The natural movement of the sacroiliac joint places stress on these implants, making them vulnerable to rotation, backing out of the screw, and ultimately failing to maintain joint stability.

### SUMMARY OF THE INVENTION

Embodiments of improved spinal implants for use in fusion procedures, such as sacroiliac fusion procedures, are provided. The implant has an overall configuration for providing mechanical stability when inserted into an anatomical structure(s) until fusion occurs. The implant may include structural features that provide longitudinal strength and mitigate bending. When used for sacroiliac fusion procedures, the implant is configured to lock the sacrum and the iliac together, thereby preventing spreading. In certain embodiments, the implant may comprise a multi-surfaced, such as a 6-starred shaped (hexalobular), cross-sectional configuration. The implant may further include radially dispersed, longitudinal splines. Surfaces between the longitudinal splines may include one or more openings or windows, each sized, shaped and arranged to allow for bone to grow therethrough, or to be filled with porous lattice structures for bone to affix.

Accordingly, it is an objective of the invention to provide an orthopedic implant for the spine.

It is a further objective of the invention to provide improved spinal implants for use in sacroiliac fusion procedures.

It is yet another objective of the invention to provide an implant comprising a multi-surfaced, cross-sectional configuration, designed to provide mechanical stability when inserted into an anatomical structure(s) until fusion occurs.

It is a still further objective of the invention to provide an implant comprising a multi-surfaced, cross-sectional configuration, and designed to provide structural features that provide longitudinal strength and mitigate bending.

It is a still further objective of the invention to provide an implant comprising a multi-surfaced, cross-sectional configuration and radially dispersed longitudinal splines, and designed to provide structural features that provide longitudinal strength and mitigate bending.

It is a still further objective of the invention to provide an implant comprising a multi-surfaced, cross-sectional configuration and radially dispersed longitudinal splines, and designed to prevent sacroiliac joint spreading.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

The present invention is illustrated and described herein with reference to the various drawings, in which like reference numbers are used to denote like system components/method steps, as appropriate, and in which:

FIG. 1 is a perspective view of an illustrative embodiment of an implant for use in sacral iliac (SI) surgical procedures;

FIG. 2 is a front view of the implant illustrated in FIG. 1;

FIG. 3 is a back view of the implant illustrated in FIG. 1;

FIG. 4 is a top view of the implant illustrated in FIG. 1;

FIG. 5 is a bottom view of the implant illustrated in FIG. 1;

FIG. 6 is a cross-sectional view taken along lines A-A of the implant illustrated in FIG. 2;

FIG. 7 is a cross-sectional view taken along lines B-B of the implant illustrated in FIG. 3;

FIG. 8 is a partial perspective view of the distal end of the implant illustrated in FIG. 1;

FIG. 9 is a partial perspective view of the proximal end of the implant illustrated in FIG. 1;

FIG. 10 is a perspective view of an alternative embodiment of the implant for use in sacral iliac (SI) surgical procedures;

FIG. 11 is a front view of the implant illustrated in FIG. 10;

FIG. 12 is a back view of the implant illustrated in FIG. 10;

FIG. 13 is a top view of the implant illustrated in FIG. 10;

FIG. 14 is a bottom view of the implant illustrated in FIG. 10;

FIG. 15 is a cross-sectional view taken along lines C-C of the implant illustrated in FIG. 11;

FIG. 16 is a cross-sectional view taken along lines D-D of the implant illustrated in FIG. 12;

FIG. 17 is a partial perspective view of the distal end of the implant illustrated in FIG. 10;

FIG. 18 is a partial perspective view of the proximal end of the implant illustrated in FIG. 10;

FIG. 19 is a perspective view of an alternative embodiment of the implant for use in sacral iliac (SI) surgical procedures;

FIG. 20 is a front view of the implant illustrated in FIG. 19;

FIG. 21 is a back view of the implant illustrated in FIG. 19;

FIG. 22 is a top view of the implant illustrated in FIG. 19;

FIG. 23 is a bottom view of the implant illustrated in FIG. 19;

FIG. 24 is a cross-sectional view taken along lines E-E of the implant illustrated in FIG. 20;

FIG. 25 is a cross-sectional view taken along lines F-F of the implant illustrated in FIG. 21;

FIG. 26 is a partial perspective view of the distal end of the implant illustrated in FIG. 19;

FIG. 27 is a partial perspective view of the proximal end of the implant illustrated in FIG. 19;

FIG. 28 is a partial perspective view of the proximal end of an alternative embodiment of the implant for use in sacral iliac (SI) surgical procedures;

FIG. 29 is a front view of the implant illustrated in FIG. 28;

FIG. 30 is a back view of the implant illustrated in FIG. 28;

FIG. 31 is a top view of the implant illustrated in FIG. 28;

FIG. 32 is a bottom view of the implant illustrated in FIG. 28;

FIG. 33 illustrates the implant shown in FIG. 1, illustrating rough or lattice surface locations and smooth surface locations;

FIG. 34 is a cross-sectional view taken along lines G-G of the implant illustrated in FIG. 1;

FIG. 35 is a perspective view of an alternative embodiment of the implant for use in sacral iliac (SI) surgical procedures, shown having less than six, starred shape sides;

FIG. 36 is a front view of the implant illustrated in FIG. 35;

FIG. 37 is a back view of the implant illustrated in FIG. 35;

FIG. 38 is a top view of the implant illustrated in FIG. 35;

FIG. 39 is a bottom view of the implant illustrated in FIG. 35;

FIG. 40 is a cross-sectional view taken along lines H-H of the implant illustrated in FIG. 36;

FIG. 41 is a cross-sectional view taken along lines I-I of the implant illustrated in FIG. 37;

FIG. 42 is a partial perspective view of the distal end of the implant illustrated in FIG. 35;

FIG. 43 is a perspective view of an alternative embodiment of the implant for use in sacral iliac (SI) surgical procedures, shown having less than six, starred shape sides;

FIG. 44 is a top view of the implant illustrated in FIG. 43;

FIG. 45 is a front view of the implant illustrated in FIG. 43;

FIG. 46 is a side view of the implant illustrated in FIG. 43;

FIG. 47 is a partial perspective view of the distal, or bottom end of the implant illustrated in FIG. 43; and

FIG. 48 is a partial perspective view of the proximal or top end of the implant illustrated in FIG. 43.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated. Where a structure(s) is shown in one embodiment, other embodiments may be adapted to include such structure(s).

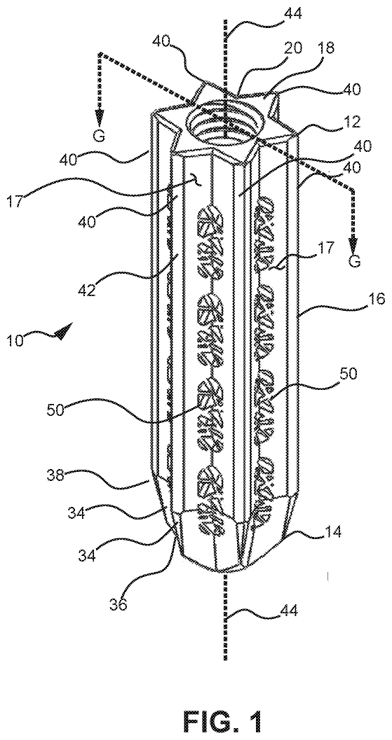

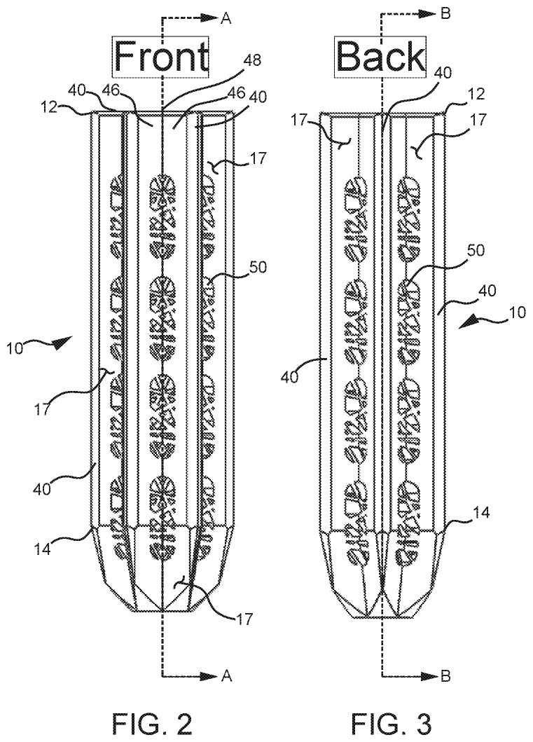

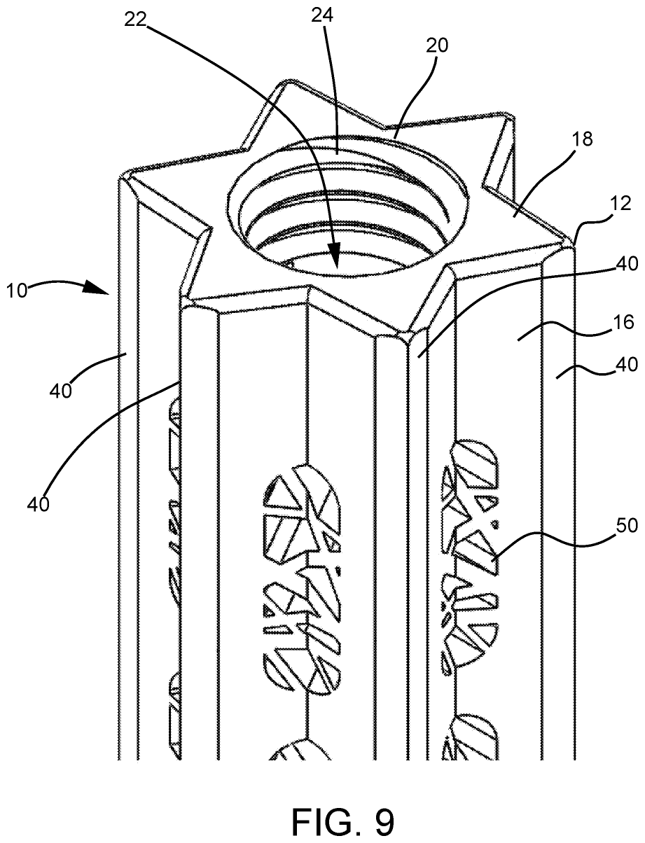

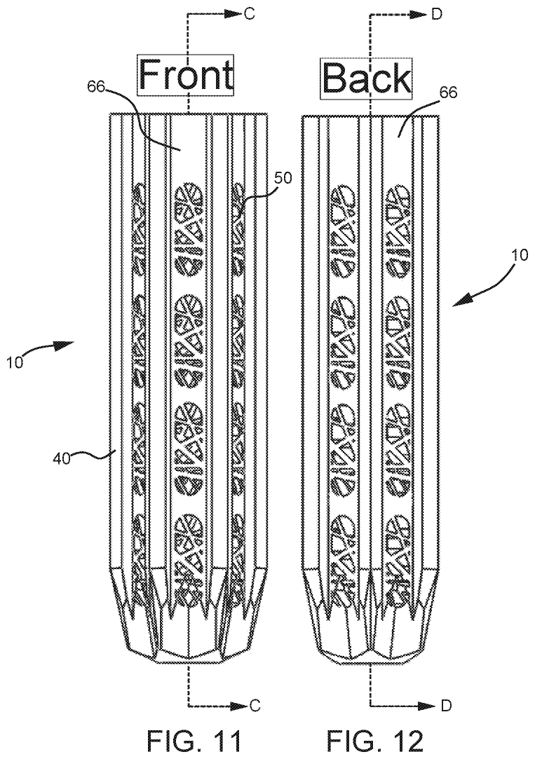

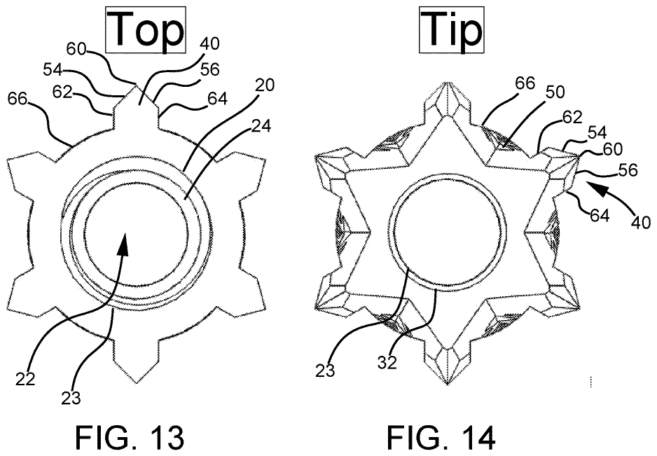

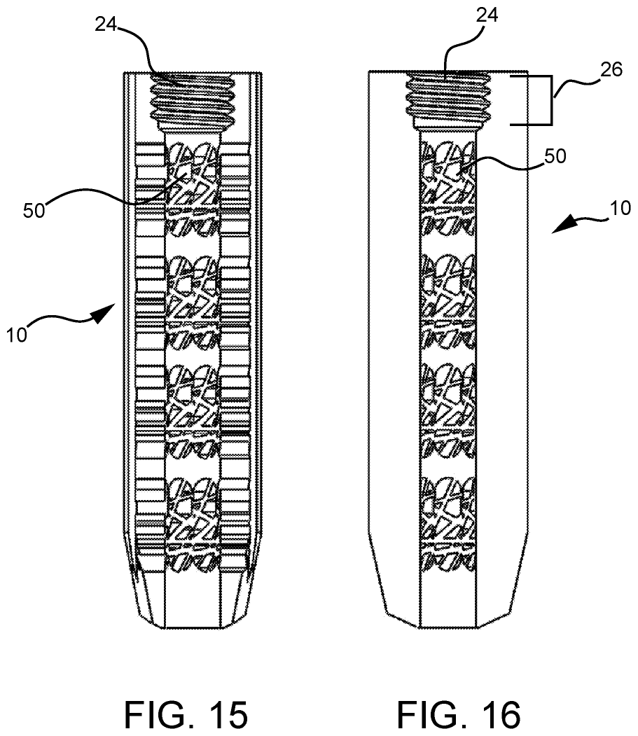

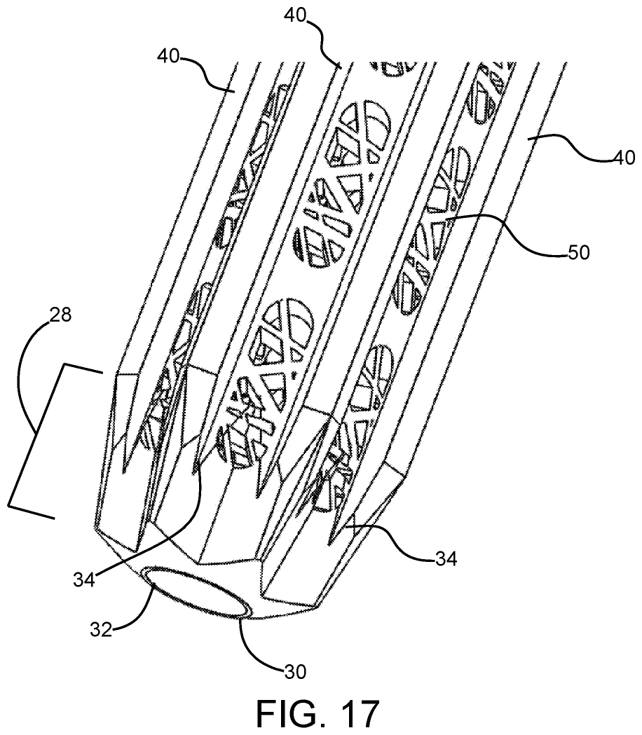

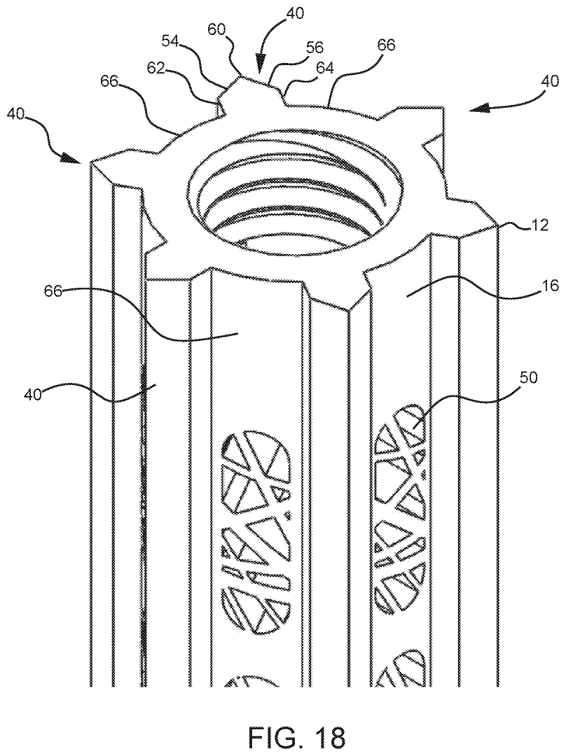

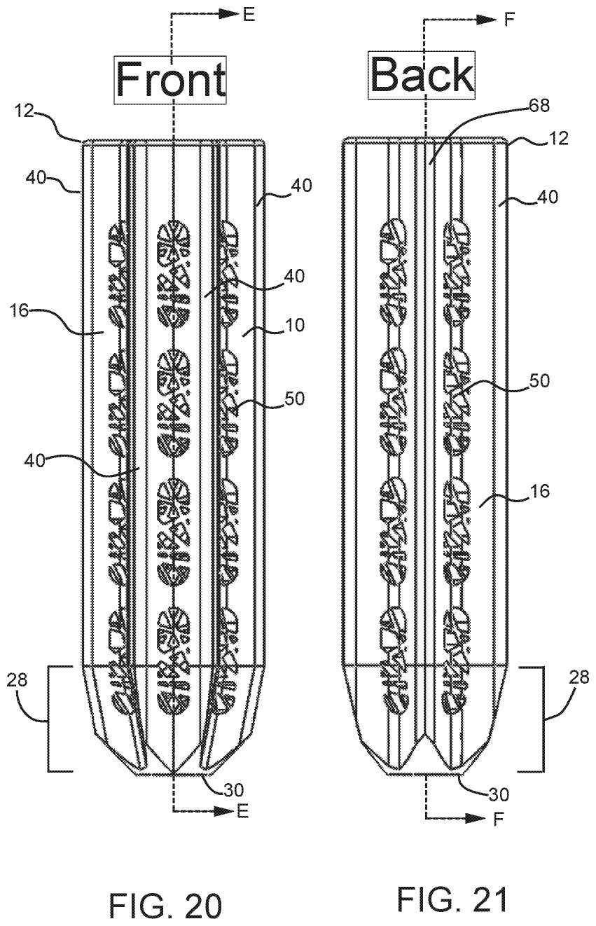

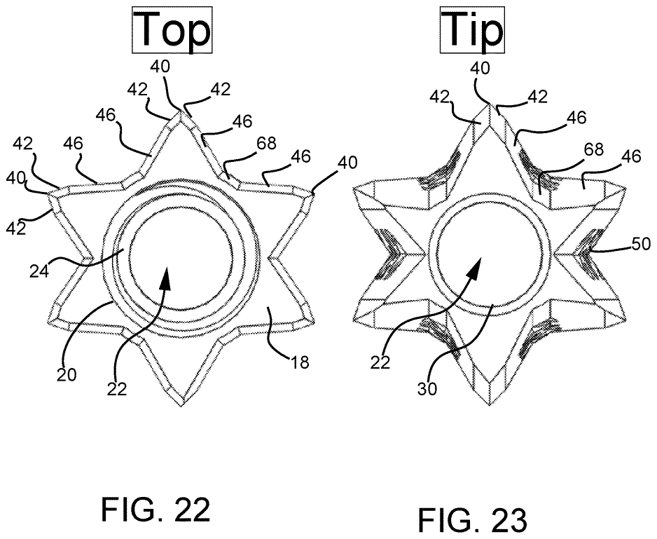

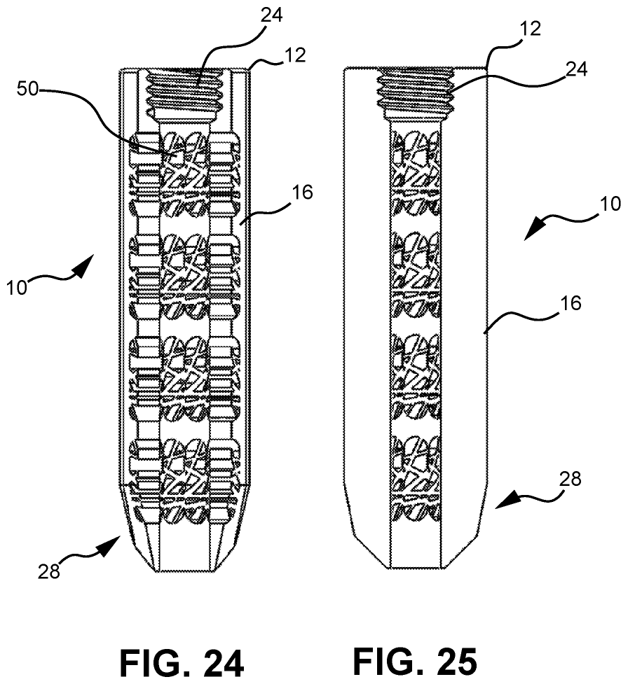

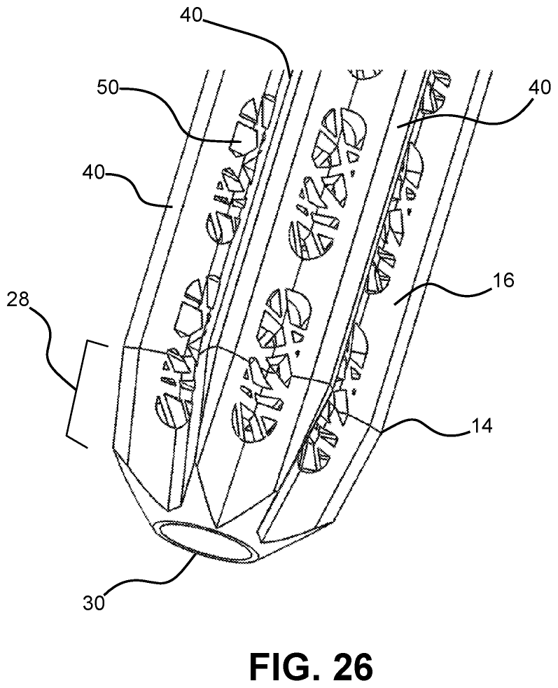

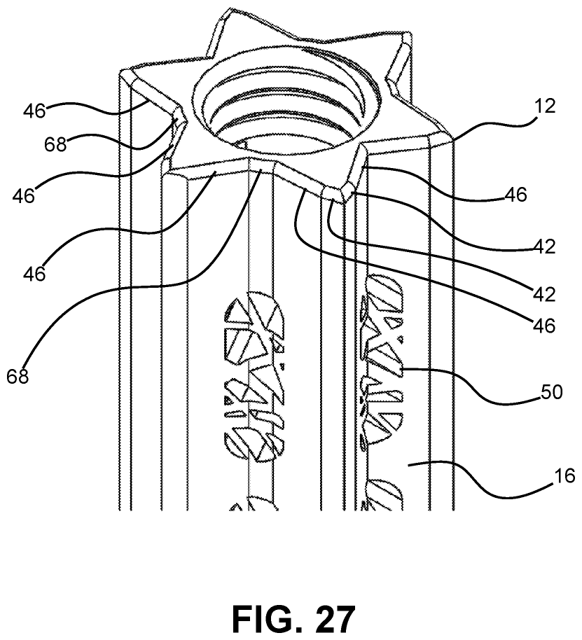

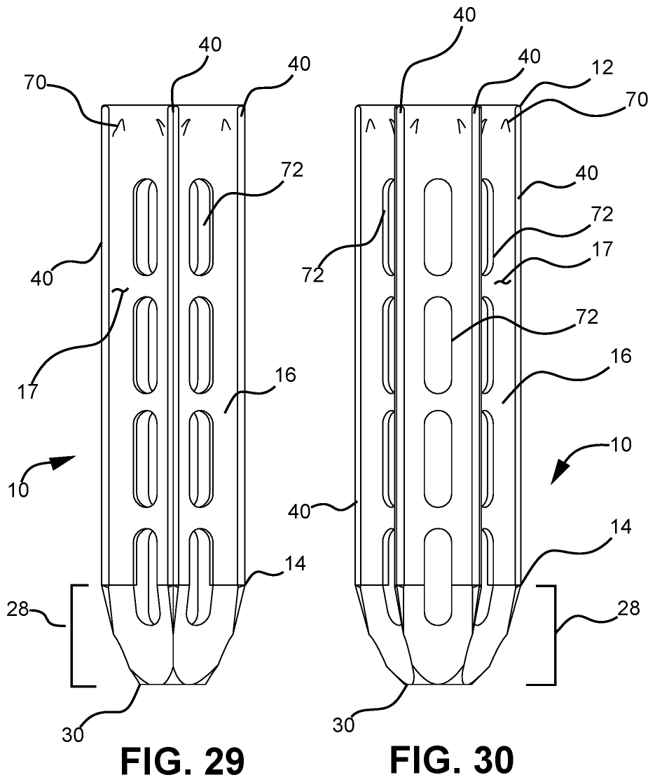

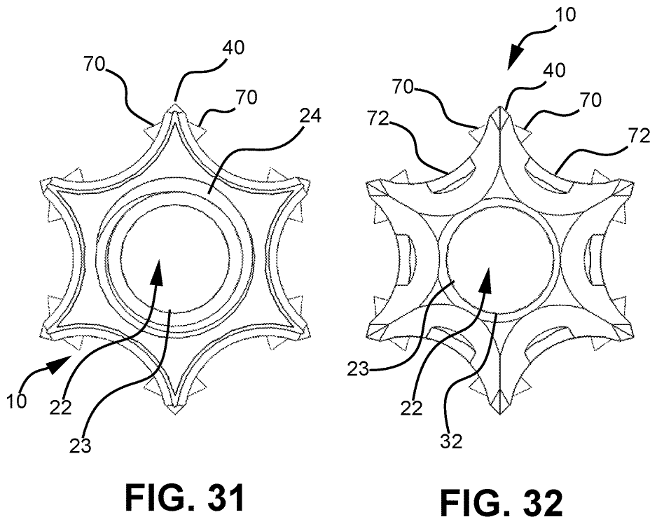

Referring to FIGS. 1-34, embodiments of a surgical implant, referred to generally as implant 10, is illustrated. While the implant 10 may be described for use in sacral iliac (SI) surgical procedures, the implant 10 may find use in other surgical procedures. The implant 10 finds use in sacral iliac (SI) surgical procedures and is designed to provide a surgeon the ability to insert or place the implant 10 using a lateral or posterior oblique trajectory when crossing from the ilium to the sacrum. In addition, the implant 10 is designed to concentrically align and affix the SI joint when using the posterior approach. Once the implant 10 is placed in position, it provides mechanical stability to the SI joint until the fusion process occurs. As needed, the implant 10 is designed to withstand the body's biomedical loads resulting from its biological response and loading. In general, the design of the implant 10 allows for use with an undersized drill to prepare a hole within the necessary anatomical structures. As such, a slight press-fit and radial compaction to the surrounding bone is provided, increasing the local density around the implant, thus enhancing the purchase of the implant 10.

Referring more specifically to FIGS. 1-9, an embodiment of the implant 10 is shown. The implant 10 may comprise a first or proximal end 12, a second or distal end 14, a body 16 separating the first end 12 from the second end 14, and an outer surface 17. The body 16 may include a multi-surfaced or multi-walled configuration, illustrated herein as a cross-sectional configuration having a six starred shape (hexalobular), see surfaces or walls 19A, 19B, 19C, 19D, 19E, and 19F. The first end 12 may include a top or upper surface 18. The upper surface 18 compromises an opening 20, preferably a centrally positioned opening, exposing an interior section or lumen 22, thus forming a cannula bore 23. When cannulated, the cannula bore 23 extends through a central portion of the interior section or lumen 22 to create a hollow channel to serve as a guide for the insertion of other instruments, such as a K-wire (Kirschner wire), drill bit, draw bar, insertion tool, or a suitable combination thereof, allowing for precise alignment, placement and securement of the implant 10. In an alternative embodiment, the interior section or lumen 22 may be formed solid or may be partially cannulated, or may include different diameter aligned cannula bores that cooperate with the different tools to aid in insertion or securement of a heavy-duty bone fastener.

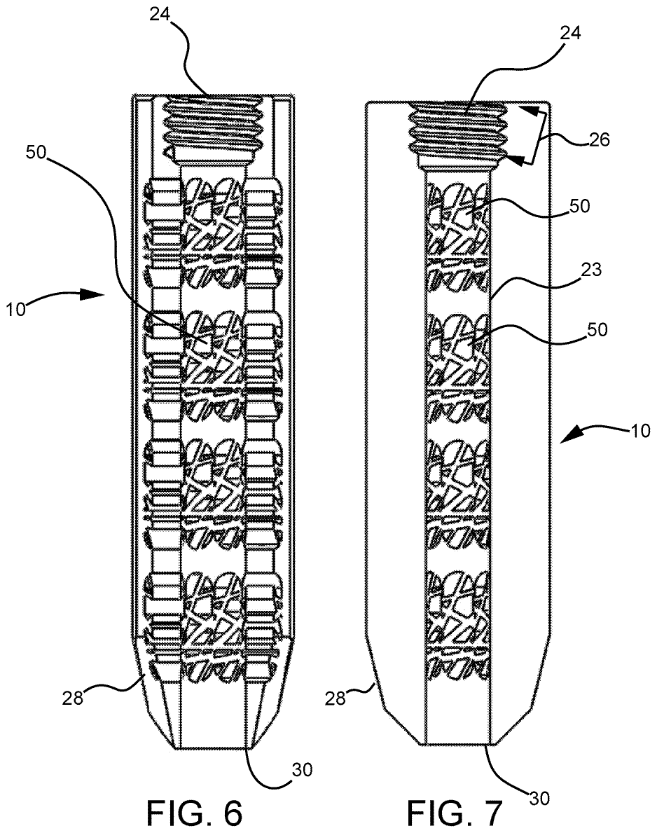

The opening 20 may include threading 24, thus forming a threaded inserter interface 26, see FIG. 7, for use in insertion or removal of the implant 10. In use, the implant 10 may be attached to an insertion device (not shown) at the threaded inserter interface 26, thus aiding the surgeon during insertion or removal of the implant 10. As shown in FIGS. 6 and 7, the threading 24 and threaded inserter interface 26 do not extend the length of the body 16. Alternatively, the implant 10 may be affixed to an insertion device without the means of the threaded inserter interface 26. Moreover, the implant 10 may be affixed using a press-fit tool. The press-fit tool can be withdrawn by pulling on the instrument following implant 10 placement, but will also hold the press-fit tool in place during delivery to the surgical field.

Since the implant 10 is designed for SI surgical procedures, the implant 10 may be made of any surgical grade material known to one of skill in the art. For example, the implant 10 may be made of cortical bone, polymers, or metals (mixed metals) such as titanium, ceramics, or combinations thereof. The implant 10 may be designed in a variety of sizes, thus allowing the surgeon to select an implant 10 that accurately and maximally fits within the bones based on the individuals' anatomical structures and/or the disease state in need of correction. In certain embodiments, the implant 10 may be constructed entirely, or in part, from a porous or material titanium (6Al-4V-Eli), such as a metal or ceramic material, to enhance its osteoconductivity and enhance its ability to bond to the bone around it. In certain embodiments, the implant 10 may be constricted using 3D printing technology. For example, the 3D printed implant 10 may be made using a Titanium Alloy (Ti-6Al-4V), using direct metal laser sintering, a dual layer organic lattice structure designed to mimic a bony trabecular structure. In this illustrative embodiment, the dual layer may have a total thickness of 1 mm. The 1 mm lattice structure may have a dual layer having a 0.5 mm pore size (micro) for the 0.5 mm thickness, and a 1.0 mm pore size (macro) for the remaining 0.5 mm thickness. In certain embodiment, this surface could be hydrophilic in nature to enable absorption and adherence of materials, such as peptides, bone morphogenetic proteins (BMPs), and stem cells.

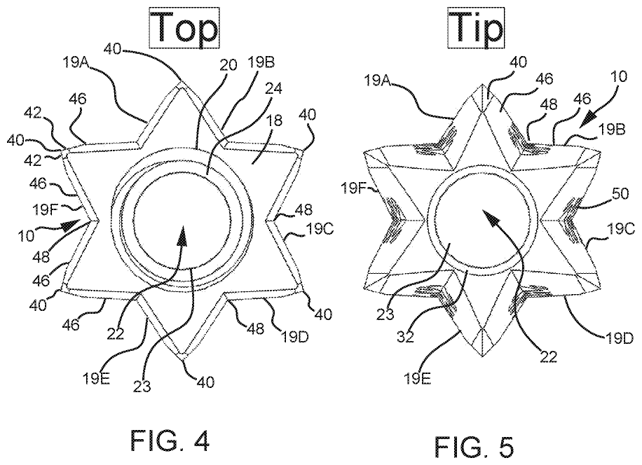

The main body 16, which provides the overall general shape of the implant 10, has a hexalobular shape, having a multi-pointed (6 points shown), star-like shape in cross-sectional view. The hexalobular shape is defined by a plurality of splines 40, each having a shaped defined by surface(s) or wall(s) 42 (shown assuming a triangular shape, see FIG. 4), and arranged along a longitudinal axis 44 of the implant 10. The longitudinal splines 40 are arranged radially about the main body 16, and spaced apart, preferably evenly spaced apart. The longitudinal splines 40 extend out and away from the main body surface 17 (or longitudinal axis 44), with the walls or surfaces (described later) forming a recessed channel. In certain embodiments, splines 40 can be configured symmetrically or non-symmetrically around the implant 10. Additionally, the splines 40 on the relative iliac or sacral side of the implant 10 can be configured to create an implant profile that is wider than the broaching or drilling tool used to create the implantation channel, yielding a configuration that resists joint distraction.

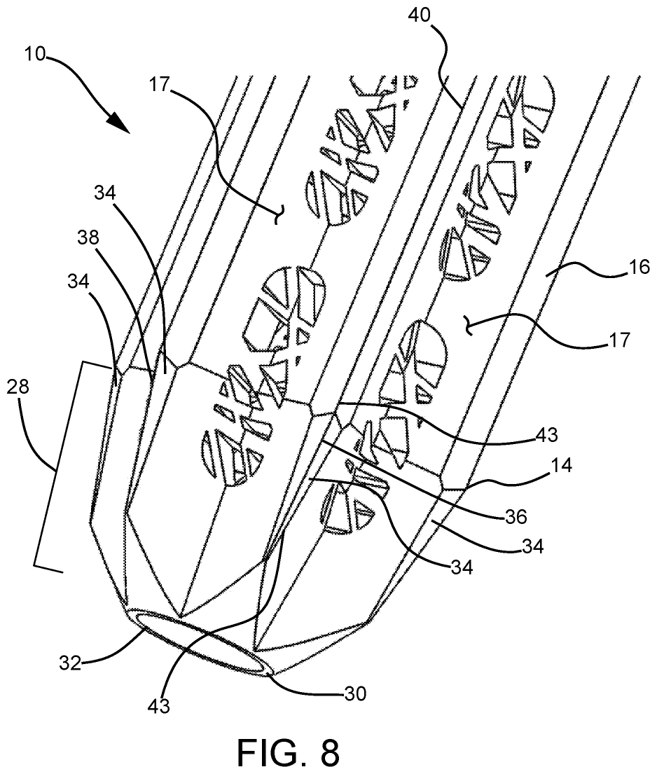

While six longitudinal splines 40 are illustrated, the implant 10 may number more or less than six, such as four, five, seven, eight, etc. The longitudinal splines 40 encompass most of the implant 10, extending from the first, proximal end 12 down to the second, distal end 14, but not extending into the tip section 28 and serve several functions. The longitudinal splines 40 arranged about or around the main body 16 increase the longitudinal strength of the implant 10 in order to mitigate bending. The arrangement may further add to the press-fitting capability. The longitudinal splines 40 may also mitigate rotation of the SI joint if the implant 10 is placed singly across the SI joint. The frontal profile of the longitudinal splines 40 are designed to mitigate the need for excessive impaction to place the implant 10 with narrow profiles and pointed surfaces 43, see FIG. 8 that begin at the proximal tip of the implant 10, and traverse longitudinally along the implant 10.

Each longitudinal spline 40 may be defined by two adjacent surfaces or walls 42. The two adjacent, diverging surfaces or walls 42 depart from the spline 40, thus forming a “triangular shaped” portion of the cross-sectional star configuration. Each longitudinal spline 40 may also include a surface or wall 46 separating each spline 40. As illustrated, the surface or wall 46 is shown as two adjacent, diverging surfaces or walls 46. Where the diverging surfaces or walls 46 of adjacent (to the left or to the right) splines 40 meet (defining the space between each longitudinal spline 40), a longitudinal joint 48 is formed. When the implant 10 is placed concentrically with the SI joint, the longitudinal splines 40 formed along the implant surface 17 act to lock the SI joint together as they form dovetail-like shapes. This arrangement transfixes the SI joint and prevents it from being able to spread. When the implant 10 is applied from a posterior approach, the implant 10 is sized to be greater in diameter than the SI joint is in breadth, thus creating a keystone effect to prevent rotation of the SI joint. This is a result of the residual ligamentous structures and dovetail being in effect, holding the SI joint from expansion while the larger diameter of the implant 10 prevents rotation since the implant 10 cannot be compressed into the narrow space.

Referring to FIGS. 1, 2, 3 and 8, the surfaces 17 between longitudinal splines 40 may include one or more macro porous lumens or openings 50, which can extend into the interior of the implant 10, see also FIG. 34. The macro porous lumens or openings 50 may be of the same size and/or shape, of varying sizes and/or shapes, or formed as clusters on the surfaces 17, and/or associated with or on the adjacent surfaces or walls 46 and/or the longitudinal joints 48. The one or more macro porous lumens or openings 50 (may also be referred to as a window(s)) function to allow large columns of bone to grow through. Alternatively, the one or more macro porous lumens or openings 50 are filled with porous lattice structures (51, see for example FIG. 34) for bone to affix thereto.

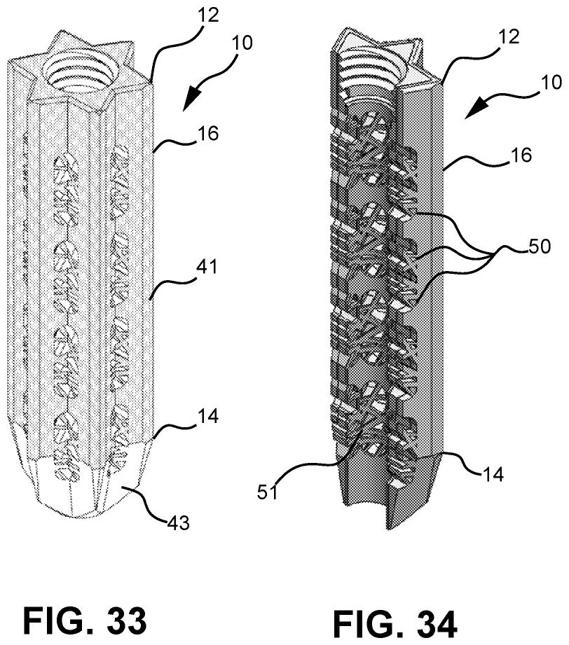

The second or distal end 14 is configured to allow for or be inserted into the anatomical structure(s). The tip section 28 of the second or distal end 14 may include a tapering terminating at a tip end 30, see FIG. 8. The tip end 30 includes a distal end opening 32. The tapering of the tip section 28 allows for ease of insertion and guides the implant 10. The longitudinal splines 40 are placed further proximal on the implant 10 to prevent misdirecting the implant 10 as it is inserted, but only grips the bone after impaction. The distal end opening 32 is sized, shaped, and positioned to receive and/or interact with one or more insertion tools. To aid in insertion, the second or distal end 14 may include one or more insertion structures 34 (may also be referred to as facets). The one or more insertion structures 34 may include distal edges having a smooth surface 36 designed to mitigate damage to the bone bed as the implant 10 is inserted, see also FIG. 1. The one or more insertion structures 34 may also include distal edges along the central and dorsal surfaces of the implant 10 for long term bone growth into the implant 10, see also FIG. 1. The implant 10 may include roughed or lattice surfaces (on the exterior surface or the interior surface, or both) along the entire length of the implant 10, such as from the proximal end 12 to the distal end 14, or any length therebetween (see FIG. 33), darkened section 41 representing the roughed or lattice surfaces; light section 43 representing the smooth surfaces). The distal tip section 28 may be smooth to ease insertion.

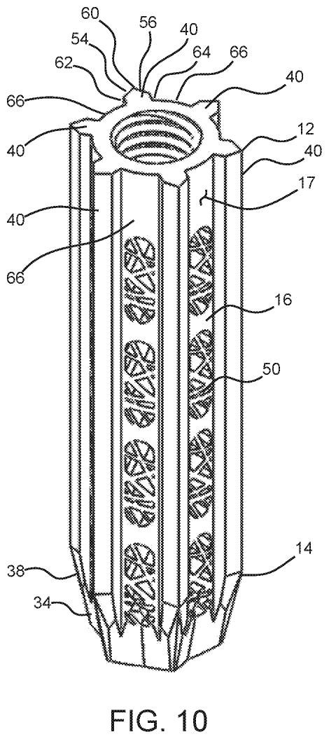

Referring to FIGS. 10-18, an embodiment of the implant 10 having different longitudinal splines 40 is shown. In this embodiment, the longitudinal splines 40 comprise two angled surfaces or walls 54, 56 converging at a surface or wall 60, thus forming a tip. A first side wall or surface 62 connects the angled surface or wall 54 to the implant body 16. A second side wall or surface 64 connects the angled surface or wall 56 to the implant body 16. A wall or surface 66 separates adjacent longitudinal splines 40. The wall or surface 66 may have a linear shape or may assume a rounded or slightly rounded shape. As the longitudinal splines 40 are designed to extend out and away from the main body surface 17, the wall or surface 66 may form a recessed channel.

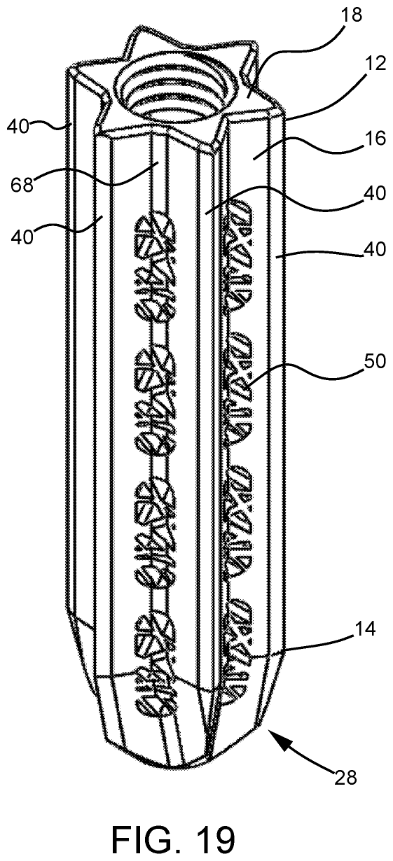

Referring more specifically to FIGS. 19-27, an embodiment of the implant 10 having a different surface structure in between adjacent longitudinal splines 40 is shown. In this embodiment, the longitudinal splines 40 are configured in the same manner described in FIGS. 1-9. In the embodiment described in FIGS. 19-27, instead of adjacent surfaces or walls 46 meeting to form the longitudinal joint 48, adjacent surfaces or walls 46 meet at a linear shaped wall or surface 68.

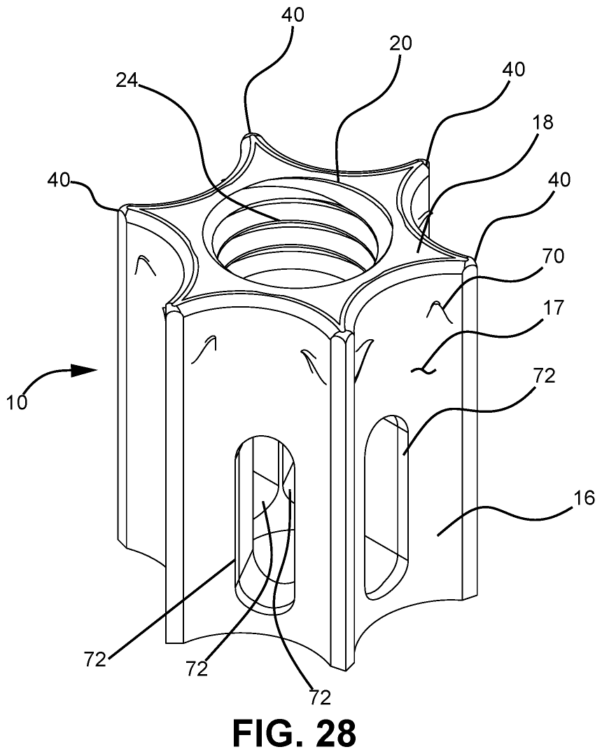

Referring more specifically to FIGS. 28-32, an embodiment of the implant 10 comprising a barbed surface is shown. The implant 10 illustrated in FIGS. 28-32 comprises many of the same structural features as the implant illustrated in FIGS. 1-9. To aid in preventing the implant 10 from migrating until the bone biologically affixes to the implant 10, the proximal end 12 may include one or more barbs 70. In addition, the implant 10 may include openings, or windows 72, cut into the surface and extending into the inner portion or lumen 22, through to an opposing side to allow larger columns of bone to grow across the implant 10, providing a long-term mechanism to affix the implant 10 in place.

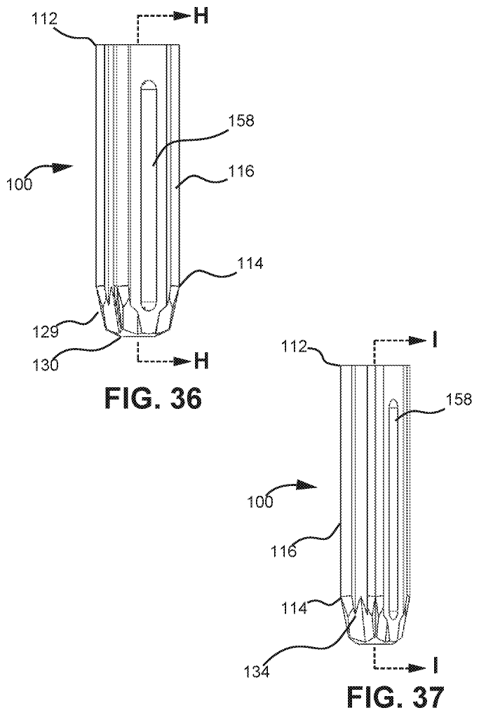

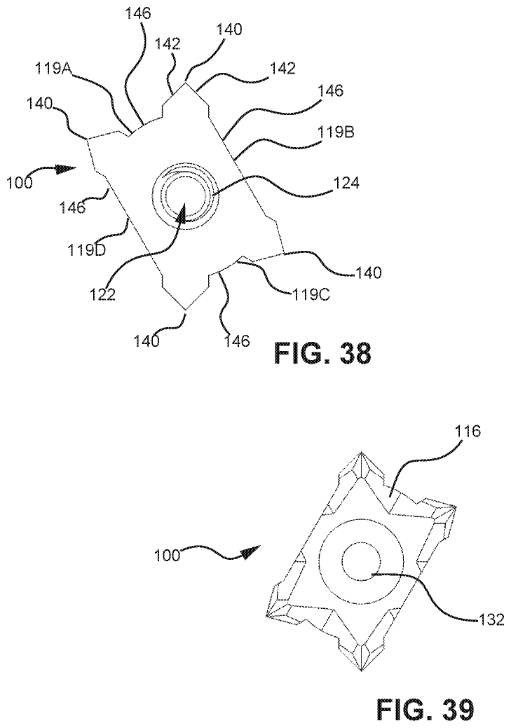

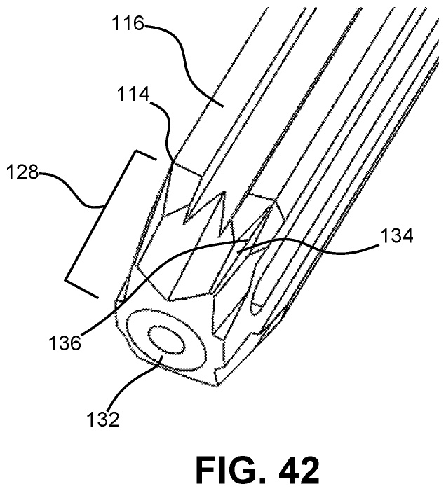

Referring to FIGS. 36-42, an embodiment of the surgical implant described above having less than a 6-starred shaped (hexalobular) cross-sectional configuration, referred to generally as implant 100, is illustrated. In the embodiment illustrated, the implant 100 is a multi-surfaced, cross-sectional configuration having a four-sided shaped sectional configuration. The implant 100 may include any feature or structural component in any combination, as described above and/or shown in FIGS. 1-35.

The implant 100 may comprise a first or proximal end 112, a second or distal end 114, a body 116 separating the first end 112 from the second end 114, and an outer surface 117. The body 116 may include a multi-surfaced or multi-walled configuration, illustrated herein as a cross-sectional configuration having four-sided shaped (quadrilobular) surfaces or walls 119A, 119B, 119C, and 119D. The first end 112 may include a top or upper surface 118. The upper surface 118 compromises an opening 120, preferably a centrally positioned opening, exposing an interior section or lumen 122, thus forming a cannula bore 123. When cannulated, the cannula bore 123 extends through a central portion of the interior section or lumen 122 to create a hollow channel to serve as a guide for the insertion of other instruments, such as a K-wire (Kirschner wire), drill bit, draw bar, insertion tool, or a suitable combination thereof, allowing for precise alignment, placement and securement of the implant 100. In an alternative embodiment, the interior section or lumen 122 may be formed solid or may be partially cannulated, or may include different diameter aligned cannula bores that cooperate with the different tools to aid in insertion or securement of a heavy-duty bone fastener.

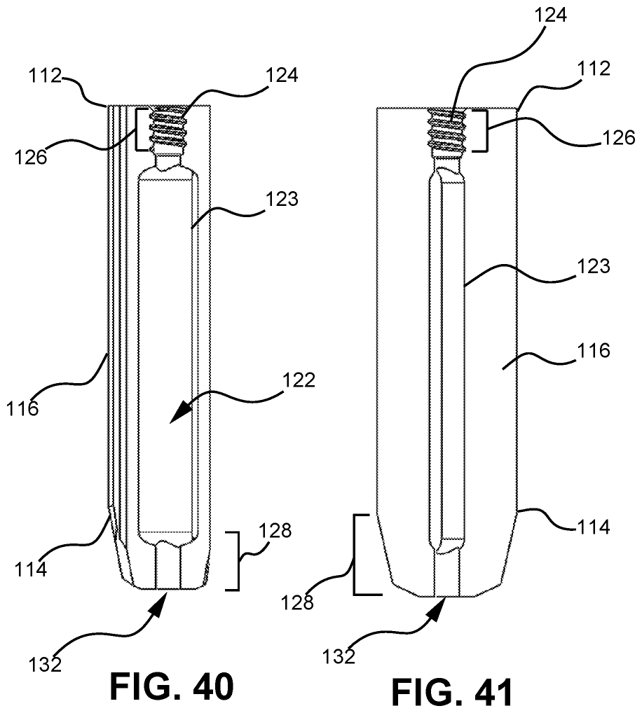

The opening 120 may include threading 124, thus forming a threaded inserter interface 126, see FIG. 40 or FIG. 41, for use in insertion or removal of the implant 100. In use, the implant 100 may be attached to an insertion device (not shown) at the threaded inserter interface 126, thus aiding the surgeon during insertion or removal of the implant 100. As shown in FIGS. 40 and 41, the threading 124 and threaded inserter interface 126 do not extend the length of the body 116. Alternatively, the implant 100 may be affixed to an insertion device without the means of the threaded inserter interface 126. Moreover, the implant 100 may be affixed using a press-fit tool. The press-fit tool can be withdrawn by pulling on the instrument following implant 100 placement, but will also hold the press-fit tool in place during delivery to the surgical field.

Since the implant 100 is designed for SI surgical procedures, the implant 100 may be made of any surgical grade material known to one of skill in the art. For example, the implant 100 may be made of cortical bone, polymers, or metals such as titanium, ceramics, or combinations thereof. The implant 100 may be designed in a variety of sizes, thus allowing the surgeon to select an implant 100 that accurately and maximally fits within the bones based on the individuals'anatomical structures and/or the disease state in need of correction. In certain embodiments, the implant 100 may be constructed entirely, or in part, from a porous or material titanium (6Al-4V-Eli), such as a metal or ceramic material, to enhance its osteoconductivity and enhance its ability to bond to the bone around it. In certain embodiments, the implant 100 may be constricted using 3D printing technology. For example, the 3D printed implant 100 may be made using a Titanium Alloy (Ti-6Al-4V), using direct metal laser sintering, a dual layer organic lattice structure designed to mimic a bony trabecular structure. In this illustrative embodiment, the dual layer may have a total thickness of 1 mm. The 1 mm lattice structure may have a dual layer having a 0.5 mm pore size (micro) for the 0.5 mm thickness, and a 1.0 mm pore size (macro) for the remaining 0.5 mm thickness. In certain embodiments, this surface could be hydrophilic in nature to enable absorption and adherence of materials, such as peptides, bone morphogenetic proteins (BMPs), and stem cells.

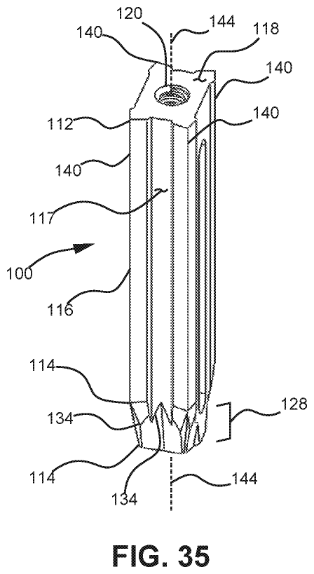

The main body 116, which provides the overall general shape of the implant 100, has a quadrilobular shape, having a multi-pointed shape (four points shown), in cross-sectional view. The quadrilobular shape is defined by a plurality of splines 140, each having a shaped defined by surface(s) or wall(s) 142 (shown assuming a triangular shape, see FIG. 35), and arranged along a longitudinal axis 144 of the implant 100. The longitudinal splines 140 are arranged radially about the main body 116, and spaced apart, preferably evenly spaced apart. The longitudinal splines 140 extend out and away from the main body surface 117, with the walls or surfaces (described later) forming a recessed channel.

The longitudinal splines 140 extend from the first, proximal end 112 down to the second, distal end 114, but not extending into the tip section 128, and serve several functions. The longitudinal splines 140 arranged about or around the main body 116 increase the longitudinal strength of the implant 100 in order to mitigate bending. The arrangement may further add to the press-fitting capability. The longitudinal splines 140 may also mitigate rotation of the SI joint if the implant 100 is placed singly across the SI joint. The frontal profile of the longitudinal splines 140 are designed to mitigate the need for excessive impaction to place the implant 100 with narrow profiles and pointed surfaces that begin at the proximal tip of the implant 100, and traverse longitudinally along the implant 100. Each longitudinal spline 140 may be separated from other splines by surfaces or walls 146.

When the implant 100 is placed concentrically with the SI joint, the longitudinal splines 140 formed along the implant surface 117 act to lock the SI joint together as they form dovetail-like shapes. This arrangement transfixes the SI joint and prevents it from being able to spread. When the implant 100 is applied from a posterior approach, the implant 100 is sized to be greater in diameter than the SI joint is in breadth, thus creating a keystone effect to prevent rotation of the SI joint. This is a result of the residual ligamentous structures and dovetail being in effect, holding the SI joint from expansion while the larger diameter of the implant 100 prevents rotation since the implant 10 cannot be compressed into the narrow space.

The surfaces 117 between longitudinal splines 140 may include one or more macro porous lumens or openings (similar to one or more macro porous lumens or openings 50) which may extend into the interior of the implant 100.

The second or distal end 114 is configured to allow for or be inserted into the anatomical structure(s). The tip section 128 of the second or distal end 114 may include a tapering section 129, terminating at a tip end 130, see FIG. 36. The tip end 130 includes a distal end opening 132. The tapering of the tip section 128 allows for ease of insertion and guides the implant 100. The longitudinal splines 140 are placed further proximal on the implant 100 to prevent misdirecting the implant 100 as it is inserted but only grips the bone after impaction. The distal end opening 132 is sized, shaped, and positioned to receive and/or interact with one or more insertion tools. To aid in insertion, the second or distal end 114 may include one or more insertion structures 134 (may also be referred to as facets). The one or more insertion structures 134 may include distal edges having a smooth surface 136 designed to mitigate damage to the bone bed as the implant 100 is inserted, see also FIG. 1. The one or more insertion structures 134 may also include distal edges along the central and dorsal surfaces of the implant 100 for long term bone growth into the implant 100. The implant 100 may include roughed or lattice surfaces (on the exterior surface or the interior surface, or both) along the entire length of the implant 100, such as from the proximal end 112 to the distal end 114, or any length therebetween (similar to that descried and shown in FIG. 33). The distal tip section 128 may be smooth to ease insertion.

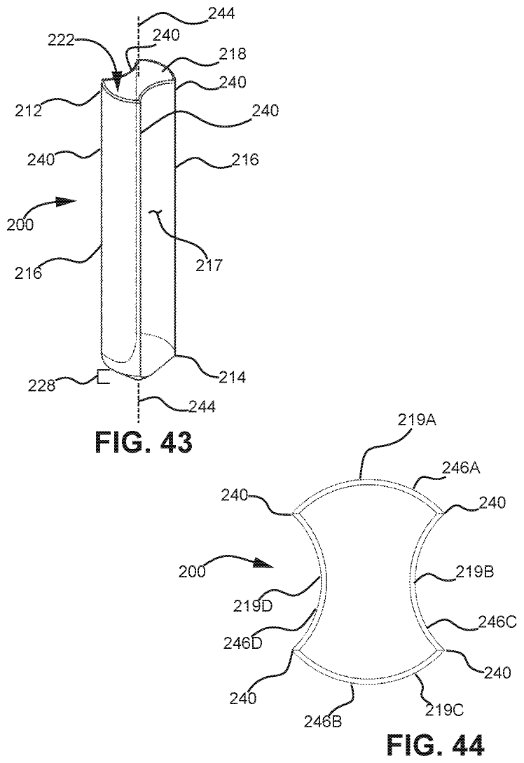

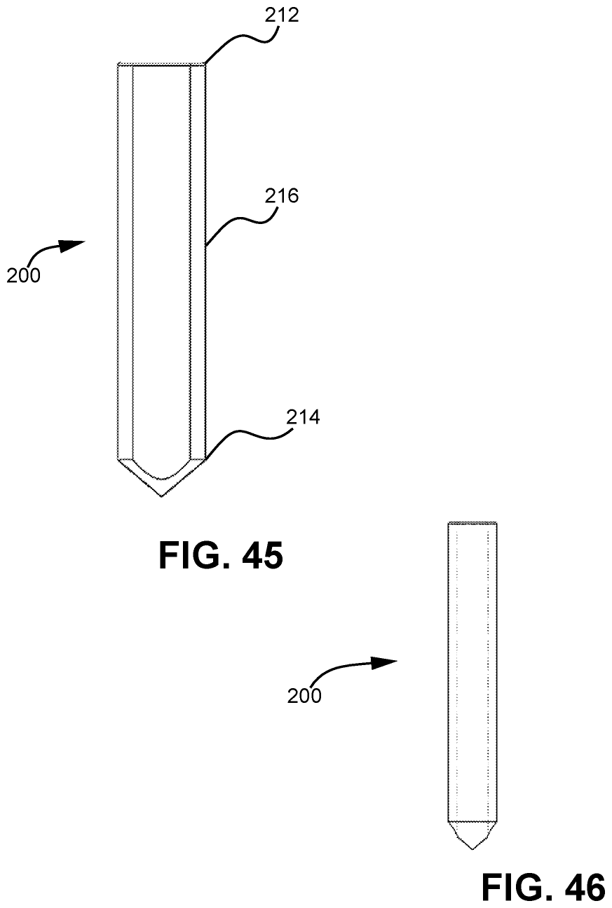

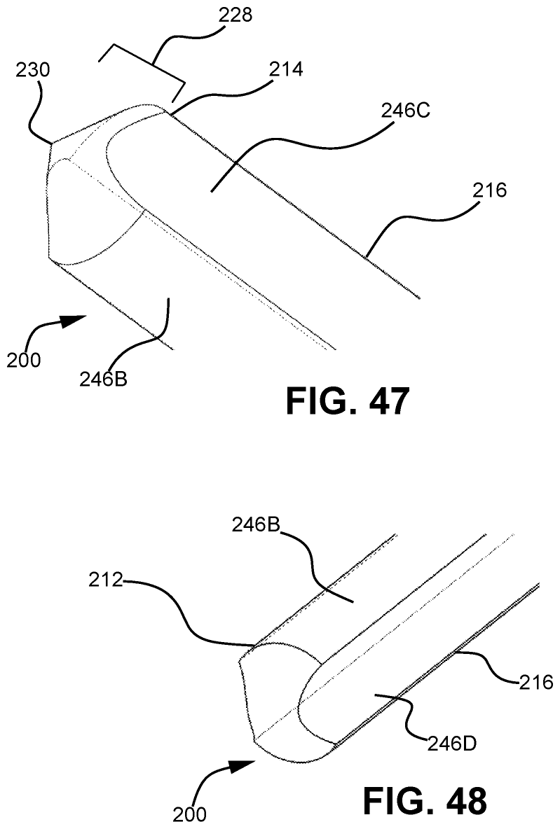

Referring to FIGS. 43-48, an embodiment of the surgical implant described above having less than a 6-starred shaped (hexalobular) cross-sectional configuration, referred to generally as implant 200, is illustrated. In the embodiment illustrated, the implant 200 is multi-surfaced, with a cross-sectional configuration having a four-sided shaped configuration. The implant 200 may include any feature or structural component in any combination, as described above and/or shown in FIGS. 1-32, implant 10 or implant 100.

The implant 200 may comprise a first or proximal end 212, a second or distal end 214, a body 216 separating the first end 212 from the second end 214, and an outer surface 217. The body 216 may include a multi-surfaced or multi-walled configuration, illustrated herein as a cross-sectional configuration having four (quadrilobular) surfaces or walls 219A, 219B, 219C, and 219D. The first end 212 may include a top or upper surface 218. The upper surface 218 may be closed or include an opening, exposing an interior section or lumen 222. Similar to implant 10 or 100, if cannulated, a cannulated bore may extend through a central portion of the interior section or lumen 222 to create a hollow channel to serve as a guide for the insertion of other instruments, allowing for precise alignment, placement and securement of the implant 200. In an alternative embodiment, the interior section or lumen 222 may be formed solid or may be partially cannulated, or may include different diameter aligned cannula bores that cooperate with the different tools to aid in insertion or securement of a heavy-duty bone fastener.

Since the implant 200 is designed for SI surgical procedures, the implant 200 may be made of any surgical grade material known to one of skill in the art. For example, the implant 200 may be made of cortical bone, polymers, or metals such as titanium, ceramics, or combinations thereof. The implant 200 may be designed in a variety of sizes, thus allowing the surgeon to select an implant 200 that accurately and maximally fits within the bones based on the individuals' anatomical structures and/or the disease state in need of correction. In certain embodiments, the implant 200 may be constructed entirely, or in part, from a porous or material titanium 6Al-4V-Eli), such as a metal or ceramic material, to enhance its osteoconductivity and enhance its ability to bond to the bone around it. In certain embodiments, the implant 100 may be constricted using 3D printing technology. For example, the 3D printed implant 200 may be made using a Titanium Alloy (Ti-6Al-4V), using direct metal laser sintering, a dual layer organic lattice structure designed to mimic a bony trabecular structure. In this illustrative embodiment, the dual layer may have a total thickness of 1 mm. The 1 mm lattice structure may have a dual layer having a 0.5 mm pore size (micro) for the 0.5 mm thickness, and a 1.0 mm pore size (macro) for the remaining 0.5 mm thickness. In certain embodiment, this surface could be hydrophilic in nature to enable absorption and adherence of materials, such as peptides, bone morphogenetic proteins (BMPs), and stem cells.

The main body 216, which provides the overall general shape of the implant 200, has a quadrilobular shape, having a multi-pointed shape (four points shown), in cross-sectional view. The quadrilobular shape is defined by a plurality of splines 240 arranged along a longitudinal axis 244 of the implant 200. The longitudinal splines 240 are arranged radially about the main body 216, and spaced apart, preferably evenly spaced apart. The longitudinal splines 240 extend out and away from the main body surface 117, with the walls or surfaces separating the splines 240 (described later) forming a relief area.

The longitudinal splines 240 extend from the first, proximal end 212 down to the second, distal end 214, but not extending into the tip section 228 with a tapered, pointed terminal end 230, and serve several functions. The longitudinal splines 240 arranged about or around the main body 216 increase the longitudinal strength of the implant 200 in order to mitigate bending. The arrangement may further add to the press-fitting capability. The longitudinal splines 240 may also mitigate rotation of the SI joint if the implant 200 is placed singly across the SI joint. The frontal profile of the longitudinal splines 240 are designed to mitigate the need for excessive impaction to place the implant 200.

In certain embodiments, the implant 200 can be manufactured with relief zones in the outer surface of the device, making the relative width and height of the device different, and creating a profile that does not match the preparation tools in order to create macroscopic fusion areas longitudinally along the outside edges of the device to enhance visualization of the fusion mass post-operatively. The longitudinal splines 240 may be separated by surfaces or walls 246A-246D, see FIG. 44. The walls or surfaces 246A and 246B assume a convex shape, while the walls or surfaces 246C and 246D assume a concave shape.

Any patents and publications mentioned in this specification are indicative of the levels of those skilled in the art to which the invention pertains.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An implant for use in Sacroiliac joint fusion procedures comprising:
a first end comprising an upper surface;
a second end;
a body separating said first end and said second end, said body defined by a multi-surfaced or multi-walled configuration; and
one or more splines arranged along a longitudinal axis of said implant body.

2. The implant according to claim 1, wherein said upper surface comprising an opening exposing an interior section or lumen.

3. The implant according to claim 1, wherein said upper surface opening is threaded.

4. The implant according to claim 1, wherein said implant body is made of a surgical grade material.

5. The implant according to claim 4, wherein said surgical grade material is cortical bone, polymers, or one or more metals.

6. The implant according to claim 1, wherein said implant body is constructed from a porous material.

7. The implant according to claim 1, wherein said splines are arranged radially about said implant body.

8. The implant according to claim 1, wherein adjacent splines are separated by one or more walls or surfaces.

9. The implant according to claim 8, wherein said one or more walls or surfaces between said splines form recessed channels.

10. The implant according to claim 1, further including one or more macro porous lumens or openings.

11. The implant according to claim 10, wherein said one or more macro porous lumens or openings are the same size or shape.

12. The implant according to claim 11, wherein said one or more macro porous lumens or openings are the same size or shape, or of varying sizes and shapes.

13. The implant according to claim 11, wherein said one or more macro porous lumens or openings are formed as clusters.

14. The implant according to claim 11, wherein said one or more macro porous lumens or openings include lattice structures for bone to affix thereto.

15. The implant according to claim 1, wherein said second end comprises a distal tip.

16. The implant according to claim 1, wherein said distal tip comprise a tapered portion, terminating at a tip end.

17. The implant according to claim 1, wherein said implant body comprises one or more barbs.

18. The implant according to claim 1, wherein said implant body comprises elongated slotted openings.
